(19) United States
(12) Patent Application Publication
(10) Pub. No.: US 2024/0081792 A1
(43) Pub. Date: Mar. 14, 2024

(54) BIOPSY-CAPABLE DERMATOSCOPE

(71) Applicant: BEHEMOTH TECHNOLOGIES LLC, Haddonfield, NJ (US)

(72) Inventors: JOSHUA FREEDMAN, HADDONFIELD, NJ (US); MARK BENDIT, NEBO, NC (US); ARIE HIRSHBEIN, PORTLAND, OR (US)

(73) Assignee: BEHEMOTH TECHNOLOGIES LLC, Haddonfield, NJ (US)

(21) Appl. No.: 18/367,205

(22) Filed: Sep. 12, 2023

Related U.S. Application Data
(60) Provisional application No. 63/405,528, filed on Sep. 12, 2022.

(51) Int. Cl. A61B 10/02; A61B 5/00; A61F 13/02

(52) U.S. Cl. CPC A61B 10/0266; A61B 5/0077; A61B 5/444; A61F 13/02; A61B 2560/0425; A61B 2560/0443; A61F 2013/00817

(57) ABSTRACT

A dermatoscope includes features to allow a practitioner to visualize a selected skin area of a patient by means of a self-contained portable tool. The same tool may be thereafter used to perform a desired biopsy of skin findings previously visualized. The same tool allows the site to be prepared for such biopsy by application of an adhesive bandage and such adhesive bandage may likewise be closed by the same tool by means of suitable structures within the dermatoscope.

### CROSS-REFERENCE TO RELATED APPLICATIONS

This application claims priority to Provisional Patent Application Ser. No. 63/405,528, the entire contents of which is incorporated herein by reference.

### FIELD

This disclosure relates to the field of dermatology and, more particularly, to hand-held medical devices for performing dermatological procedures.

### BACKGROUND

Although biopsies of skin or surface areas of patients are regularly performed for dermatological as well as other medical reasons, such biopsy procedures suffer from various drawbacks and disadvantages. The various preparatory, biopsy, and post biopsy steps of the procedures are often performed by separate tools, associated with separate medical supplies, or otherwise are performed inefficiently and are time consuming. In the case of punch biopsies, for example, there is often a need to have multiple tools and consumables, often three tools, a suture, and a bandage, separately manipulated at separate times to perform such dermatological biopsy procedures.

The inefficiency and numerous steps not only have drawbacks for the practitioner but for the patient as well, whose anxiety may be unnecessarily increased by virtue of the multiple steps and multiple procedures to which such patient is being subjected.

It would be desirable to overcome the various drawbacks and disadvantages of the procedural methods required by other medical devices associated with biopsy procedures.

### SUMMARY

In one suitable implementation of the present disclosure, a biopsy-capable dermatoscope comprises a portable, self-contained apparatus which has a head, a handle extending from the head, and which makes use of a removably insertable, cartridge for performing dermatological procedures on a selected skin area of a patient. The head is configured to have one end placed in operative contact with the patient's skin by means of the practitioner grasping the handle extending from such head. The dermatoscope has features to permit visualization of the selected skin area and includes one or more manually actuatable controls to perform still other dermatological procedures with the same apparatus, such preoperative biopsy site selection within the cross polarized visual field, preoperative skin preparation and tensioning via bandage-clip application, biopsy of the visualized skin area, collection of the biopsy sample, and post biopsy wound closure.

To the foregoing ends, the head has a viewing opening which defines a viewing area, as well as a lens group disposed across the viewing opening and a light source located relative to such viewing opening so as to illuminate the selected skin area using polarized and non polarized, visible or ultraviolet LEDs. In addition to the visualization features, the dermatoscope has an adhesive bandage-clip assembly and a blade assembly, both disposed within the cartridge and suitably connected to manual controls so that such assemblies can perform the associated procedures when selected by the practitioner. A crimping assembly is also associated with the dermatoscope, the crimping assembly having a bandage closure mechanism and a pair of arms. The crimping assembly may be manually actuated by suitable controls so that the arms act upon the adhesive bandage clip assembly after performance of the biopsy to close an adhesive bandage over the wound site.

In other suitable implementations, the blade assembly is configured into an open-boat shape with a pair of convex sides terminating in convex, upper edges. As such, the blade defines an open volume to collect the biopsy sample after it has been cut by the sufficiently sharp upper edges of the blade. The blade assembly is configured so that manual actuation by the practitioner while holding the dermatoscope in operative contact with the skin rotates the blade through an appropriate arc to perform the desired biopsy, whether a tangential biopsy, incisional biopsy, excisional biopsy, or other desired biopsy of appropriate length, depth, and location on the desired skin area.

In still other possible implementations, the blade assembly is located within the cartridge and within the head into which the cartridge has been received, so that the cutting surfaces of the blade perform the desired biopsy at a selected incision depth. In one possible implementation, the blade rotates about a mounting axis which can be thought of as being located at an operative distance from the skin area to be biopsied, whereas the surface of the head in contact with the skin is spaced therefrom by a contact distance. The operative distance is greater than the contact distance such that when the head is appropriately positioned in operative contact with the skin, the difference between the operative distance through which the blade swings and the contact distance at which the head is held forms a difference corresponding to the desired incision depth.

In still other possible implementations, the blade assembly is positioned within the cartridge so that, when the cartridge is removably inserted into the head, the blade assembly does not excessively obstruct visualization of the skin area through the viewing opening. The blade assembly can either be rotated into a stowed position out of view or can be translatably or slidably moved in or out of the viewing area. As such, when performing the biopsy procedure after visualization, the blade may be either rotated across the viewing opening to perform the biopsy or slid from out of view and into view at which point the blade can thereafter be rotated to perform the biopsy.

The dermatoscope includes features that apply a suitable adhesive bandage to the selected skin area in preparation for performing the biopsy. Additional features of the dermatoscope close such adhesive bandage over the incision site to close the wound, including imparting appropriate eversion to the edges of the incision. In one suitable implementation, an adhesive bandage-clip assembly is disposed within the cartridge in operative proximity to the selected skin area, such as toward the distal side of the cartridge. Thereafter, suitable orthogonal force, either by manipulation of the dermatoscope itself or by actuation of a suitable control, can depress the adhesive bandage clip assembly from its starting position within the cartridge so that it adheres inferiorly to the skin, while still bound at its lateral aspects within the cartridge, thereby affixing the head of the device in the desired operative position on the patient.

The adhesive bandage thus applied may have an aperture sized so that the blade and corresponding incision stay within the boundary of such aperture while performing the biopsy. Thereafter, the dermatoscope may include suitable features, such as the crimping assembly, which has arms configured to close the adhesive bandage clip assembly and seal such bandage over the incision site to appropriately close the associated wound.

In one suitable implementation, the adhesive bandage-clip assembly makes use of a pair of flanges which are urged inwardly by the arms of the crimping assembly. The arms of the crimping assembly may have fingers, one of which urges forward one portion of the flange to close the wound and a second finger interacts with another portion of the flange which encourages upward force on the adhesive bandage to cause eversion at the incision site.

In still other implementations, the dermatoscope of this disclosure may include a set of cartridges, each of the cartridges containing therein a corresponding blade assembly having a set of blade dimensions with associated clinical characteristics. In this way, the practitioner may select a certain cartridge having blade characteristics appropriate for the desired biopsy to be performed. The size of the bandage-clip assembly housed within a given cartridge would also be scaled to the size of that particular blade cut commensurately.

The dermatoscope of this disclosure may be used to perform any number of dermatological procedures previously performed by separate medical instruments. In one such procedure, the head of the dermatoscope is brought into operative proximity to a skin area to be visualized. The same dermatoscope in the same operative position may be manipulated to apply a suitable adhesive bandage to the desired skin area in anticipation of one or more further procedures. After application of the adhesive bandage, the head is maintained in the same operative location by virtue of the of the bandage-clip assembly adhering inferiorly to the skin, and laterally to the device. The dermatoscope may be manipulated to perform the biopsy by bringing the blade assembly into contact and performing an incision in the selected skin area. Thereafter, by means of the same dermatoscope, different features may be actuated, such as the adhesive bandage clip assembly, to close the incision site. The previous action of the blade assembly and its open-boat configuration allow collection of the biopsied skin or tissue sample within the cartridge associated with the dermatoscope. The cartridge can thereafter be removed, the sample suitable removed from the cartridge, and the blade assembly and other components within the cartridge, or even the cartridge itself in some implementations, may be suitably disposed of, such as in a medical waste container.

These and other features of this disclosure will be more fully appreciated by reference to the drawings herein, which are as described below.

### BRIEF DESCRIPTION OF DRAWINGS

FIG. 1 is a side, elevational view of one possible implementation of a biopsy-capable dermatoscope according to the present disclosure;

FIG. 2 is a cross-sectional view of a dermatoscope according to the present invention, taken along line 2-2 of FIG. 1;

FIG. 3 is a cross-sectional view of another possible implementation of a dermatoscope according to this disclosure, taken along line 3-3 of FIG. 2, with a cartridge inside;

FIG. 4 is a bottom plan view of the dermatoscope shown in FIGS. 1 and 2;

FIG. 5 is an isometric view of one potential blade assembly according to the disclosure;

FIG. 6 is a partial, cross-sectional view of the blade assembly of FIG. 5 shown in association with an implementation of a cartridge according to the present disclosure, taken along line 6-6 of FIG. 3;

FIG. 7 is another partial, cross-sectional view of an implementation of a cartridge for a dermatoscope according to the present disclosure, taken along line 7-7 of FIG. 6;

FIG. 8 is a top plan view of one suitable implementation of an adhesive bandage-clip assembly suitable for use as part of a dermatoscope according to the present disclosure, in an open position;

FIG. 9 is a top plan view of the adhesive bandage-clip assembly of FIG. 8, shown in a closed position;

FIG. 10 is a side elevational view of an adhesive bandage-clip assembly according to the present disclosure;

FIG. 11 is a partial, schematic, isometric view of one potential crimping assembly in accordance with suitable implementations of the present disclosure;

FIG. 12 is another suitable implementation of a dermatoscope according to the present disclosure, including a cartridge with blade assembly shown therein schematically and in side elevation view;

FIG. 13 is another side elevational view of the implementation shown in FIG. 12, showing the blade assembly according to the present disclosure in an operative position;

FIG. 14 is an isometric view of another implementation of a dermatoscope according to the present disclosure;

FIG. 15 is an isometric view of a blade assembly according to the implementation shown in FIG. 14;

FIG. 16 is an isometric view of one possible blade for use in the implementation shown in FIGS. 14 and 15;

FIG. 17 is an isometric view of one possible implementation of a bandage-clip assembly for use in any of the implementations of the present disclosure;

FIG. 18 is an isometric, sectional view of certain features of a crimping assembly and bandage-clip assembly according to the present disclosure;

FIG. 19 is another view of a suitable implementation of a bandage-clip assembly and crimping assembly shown as a cutaway isometric view; and

FIG. 20 is a side-elevational, partly schematic view of another implementation of a dermatoscope according to the present disclosure and the blade assembly thereof.

### DETAILED DESCRIPTION

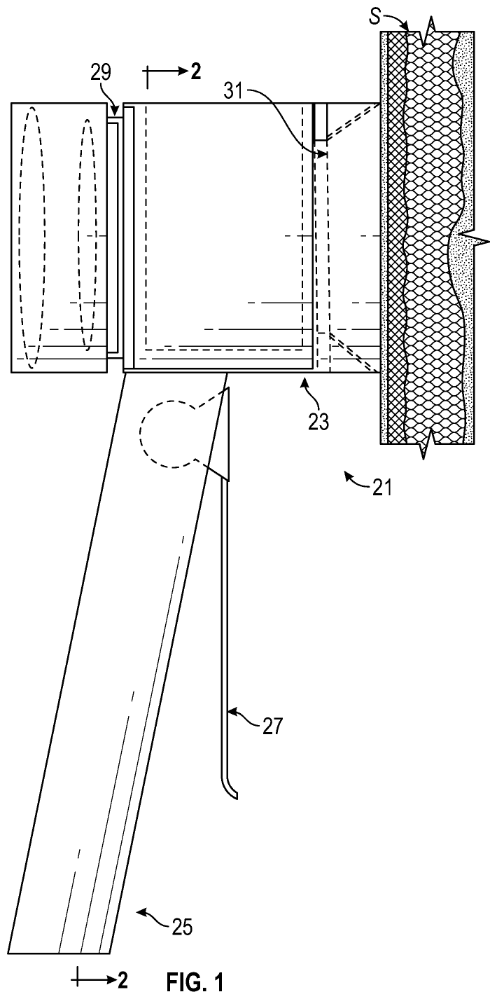

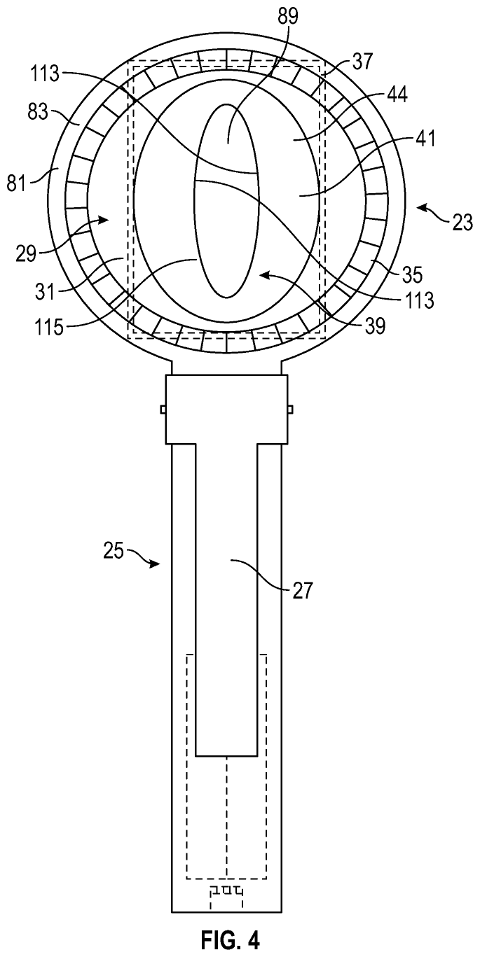

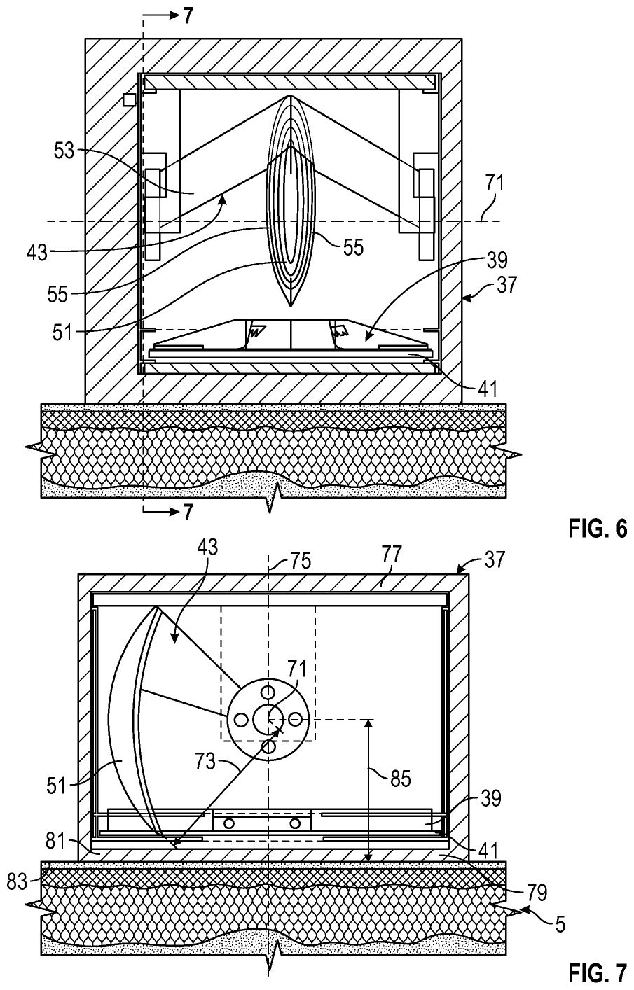

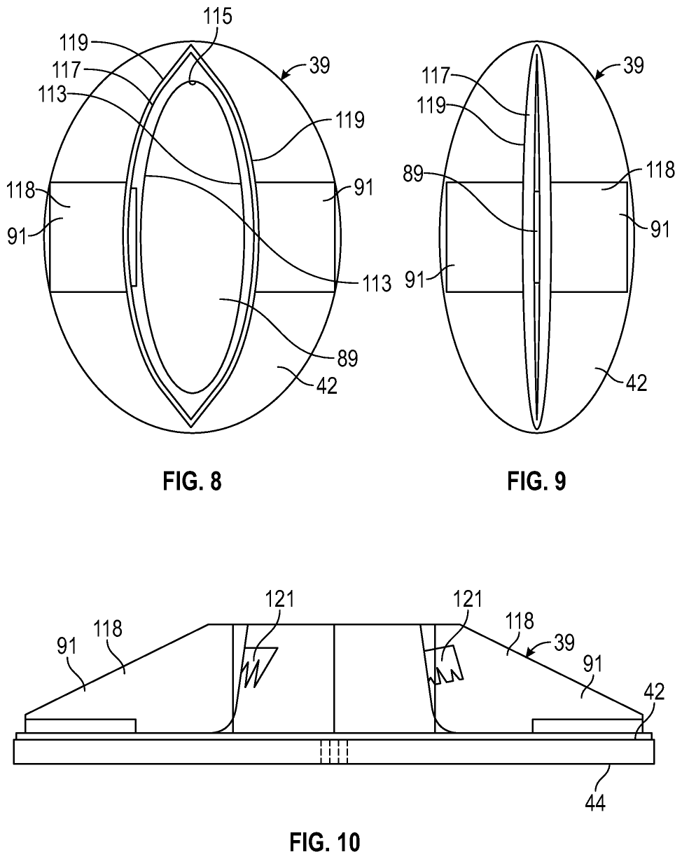

Referring now to the drawings, and in particular to FIGS. 1-11 thereof, according to certain implementations of the present disclosure, a biopsy-capable dermatoscope 21 is shown in various views to better understand its assemblies, structures, and related operations. In the illustrated implementations, biopsy-capable dermatoscope 21 is designed for a dermatologist, clinician, practitioner, or other medical personnel to use on a patient in connection with visualization, biopsy, excision, or other related dermatological procedures. To that end, dermatoscope 21 incorporates elements, features, and related assemblies to perform multiple functions typically accomplished by either separate instruments or separate steps, such as visualization of skin areas, potential biopsy thereof, excision site preparation and excision closure.

The foregoing operations of dermatoscope 21 are accomplished by suitable controls, preferably manually actuatable controls. It will be appreciated that manually actuatable may be defined to include not only movement of components by mechanical means such as levers, thumb wheels, grips, handles and the like, but manually actuatable buttons or other electronically assisted operations initiated by manually depressing buttons or other manual actions. These manually initiated operations may utilize one more types of electromechanical actuators to produce the desired mechanical outcome. To that end, biopsy-capable dermatoscope 21 may include portable power, such as batteries, which may be rechargeable. In addition or alternately, biopsy-capable dermatoscope 21 may be powered by a suitable power cord, power being provided through a suitable interconnect, such as a USB port or other electrical connection.

Dermatoscope 21 thus includes a handle 25 for grasping by the practitioner, the handle 25 having manually actuatable controls, shown schematically at 27, for performing various dermatologically related procedures, such as visualization and biopsy, skin preparation, and wound management and closure. Connected at one end of handle 25 is a head 23, head 23 being configured so that it can be placed in operative contact with a patient's skin s. The locations where the practitioner accesses controls 27 may be varied based on any number of considerations, including ergonomics, need for tactile feedback, and granularity of control. Likewise such considerations may determine the mode and manner of interconnection between buttons, levers and wheels for actuation, as well as the mechanical or electromechanical designs thereof, associated with controls 27 on the one hand, and the assemblies and associated functions corresponding to such controls.

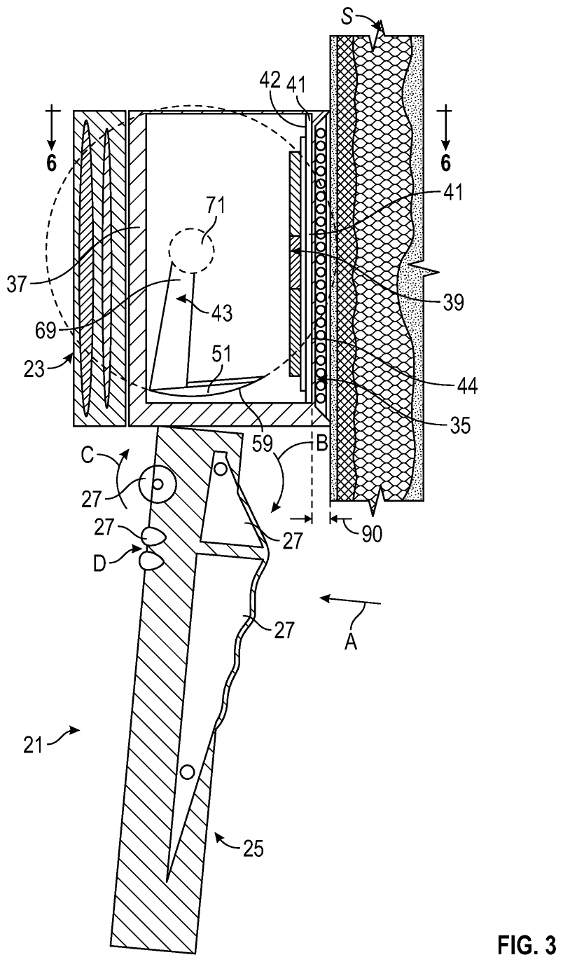

As such, by way of exemplary implementation shown in FIG. 3, controls 27 may include a pair of raised grip levers actuatable by compressive force in the direction of arrow B, a thumbwheel, dial or similar rotary component actuatable by rotation in the direction of arrow C (and the reverse), and electromechanical switches, buttons, and the like actuated by compression in the direction of arrow D. In the illustrated embodiment, the access to controls 27 occurs at locations in handle 25, and suitable electrical or mechanical interconnections respond to such actuation by causing functions to occur in head 23. Other variations are possible, including mounting certain controls elsewhere than on handle 25, such as through additional components disposed on or otherwise accessible from head 23.

Head 23 has portions defining a viewing opening 29 disposed in head 23 and defining a viewing area 31. Viewing area 31 is associated with a lens or lenses 33 disposed across viewing opening 29 to enable the practitioner to look through lenses 33 and see skin in viewing area 31. To further assist in such visualization, a light source 35 is located relative to viewing opening 29 to illuminate a selected skin area of the patient when head 23 is brought into a desired operative position on the patient, such as shown in the figures, including FIG. 1 thereof. The light sources may include polarized and nonpolarized LEDs, within the visible and ultraviolet spectrum.

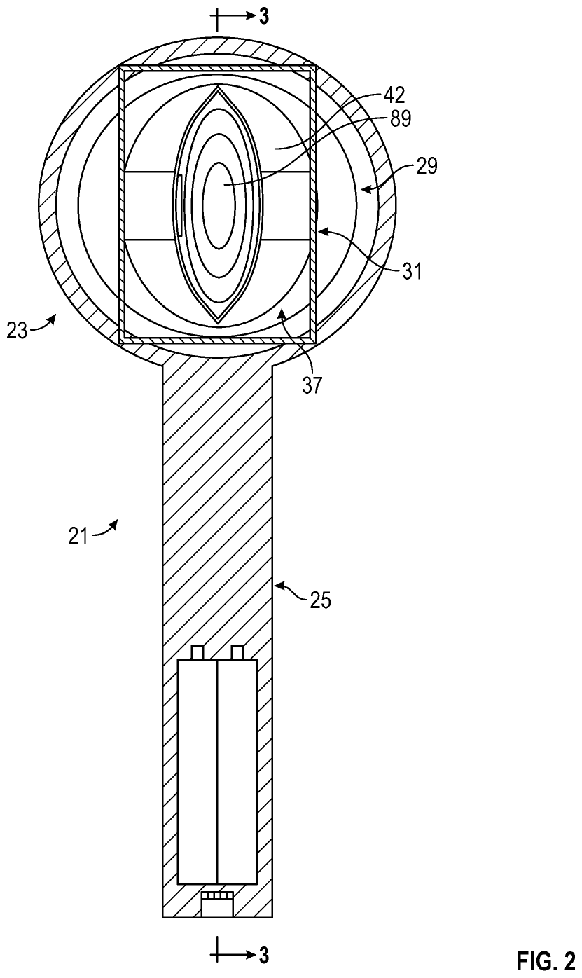

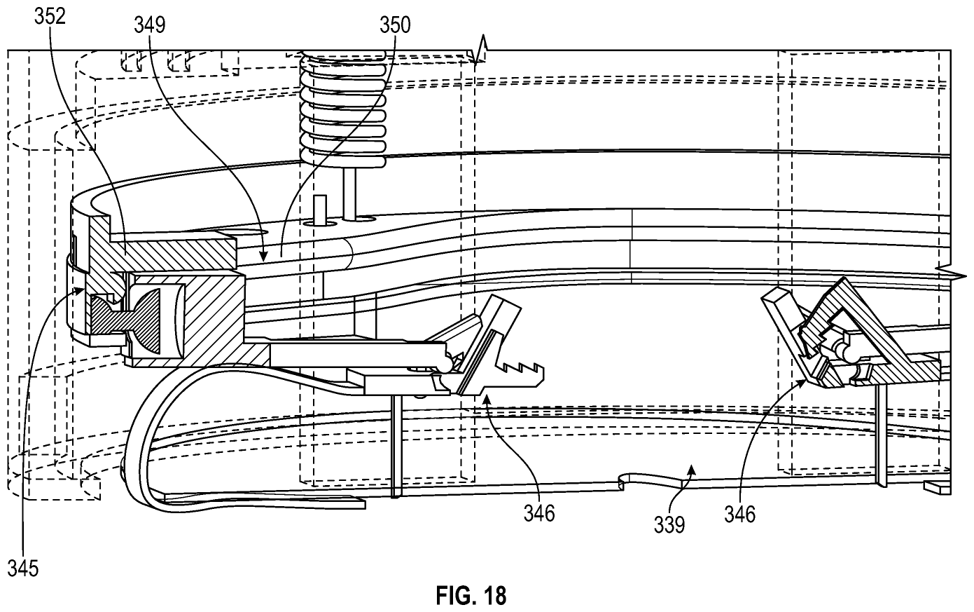

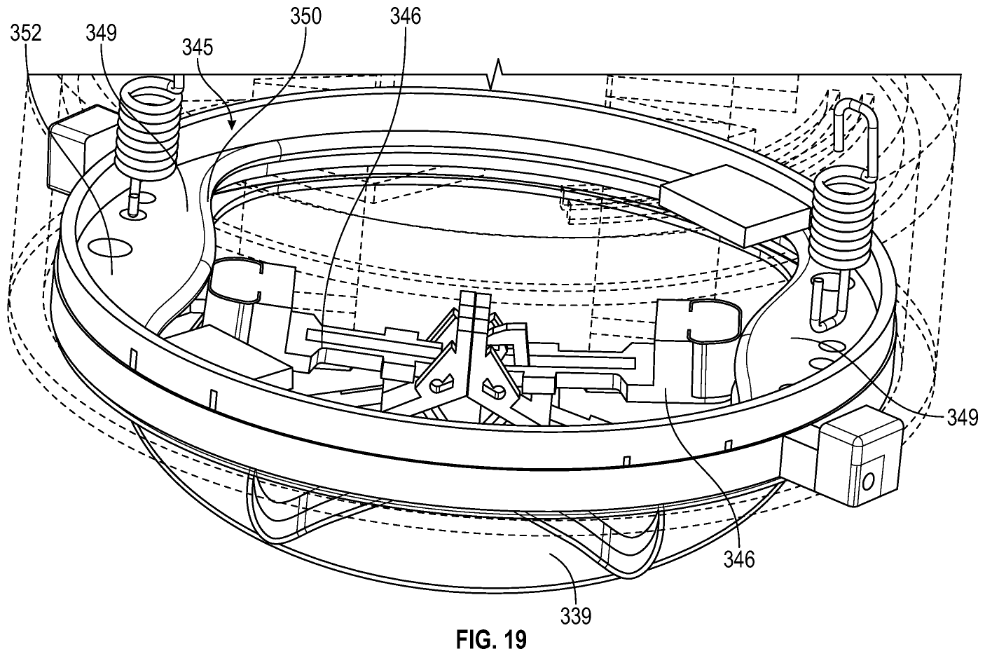

In certain implementations, dermatoscope 21 may be equipped with a disposable cartridge 37 which is removably insertable into a corresponding aperture or other area of head 23 (FIG. 2). Disposable cartridge 37 may be configured to contain therein various one-use-only components associated with the dermatological procedures contemplated for dermatoscope 21. For example, disposable cartridge 37 may have an adhesive bandage-clip assembly 39 disposed within it and positioned in operative proximity to the selected skin area when head 23 is brought into the desired operative position on the patient. Once in position above the selected skin area, the user tangentially applies a small amount of tension to stretch the skin with their free hand, and then actuates an initial mechanism which depresses either the cartridge, or the bandage-clip assembly 39 within the cartridge, to travel a very short distance from just above the inferior margin of the device, to just a few millimeters below that margin. As the adhesive bandage 41 presses downward against the skin, a slight upward displacement force on the handpiece will inform the user the action is complete. The handpiece is now adhered to the skin via the adhesive bandage-clip assembly 39 which is connected to the cartridge, which assures the user they will only biopsy the skin that is visibly affixed within the bandage aperture as seen through the viewer. Adhesive bandage-clip assembly 39 has an adhesive bandage 41 releasably secured relative to cartridge 37 to selectively separate adhesive bandage 41 from head 23 and onto the selected skin area after the biopsy and crimping actions have been completed. The depression, or downward displacement of the cartridge, or adhesive bandage-clip assembly 39 may be accomplished by suitable actuation of a manually actuatable control 27 which could accomplish this downward displacement by the mechanism shown in FIGS. 18, and 19 wherein part 352 plunges downward holding one embodiment of the bandage clip.

To perform biopsy procedures, dermatoscope 21 is equipped with a blade assembly 43 mounted within cartridge 37. Like adhesive bandage-clip assembly 39, blade assembly may be a single-use-only component. Alternatively, all or portions of blade assembly 43 may be reusable. Blade assembly 43 is movable to perform the desired biopsy incision in response to manually actuation of one of the manually actuatable controls 27. In the illustrated embodiments, blade assembly 43 is subjected to various movements, including translation and/or rotation. Blade assembly 43 may be selectively movable from a first stowed position outside a majority of viewing area 31, so as not to obstruct viewing area 31 during visualization procedures, to a second, operative position, where blade assembly 43 is able to contact or in fact contacts the skin area to perform a biopsy incision thereof.

As shown in FIGS. 1-11, the stowed position of blade assembly 43 involves keeping the majority of the blade assembly 43 to one side of cartridge 37 substantially outside of viewing area 31 and, in response to suitable actuation of the corresponding controls 27, rotating blade assembly 43 from such stowed position at the side so that blade 51 moves through an arc to a more central location within of cartridge 37 to contact the selected skin area. Controls 27 may be actuated to rotate blade assembly 43 by imparting rotation to transverse-axially-mounted connectors 101, which connectors 101 in turn rotate blade assembly 43 through struts 69.

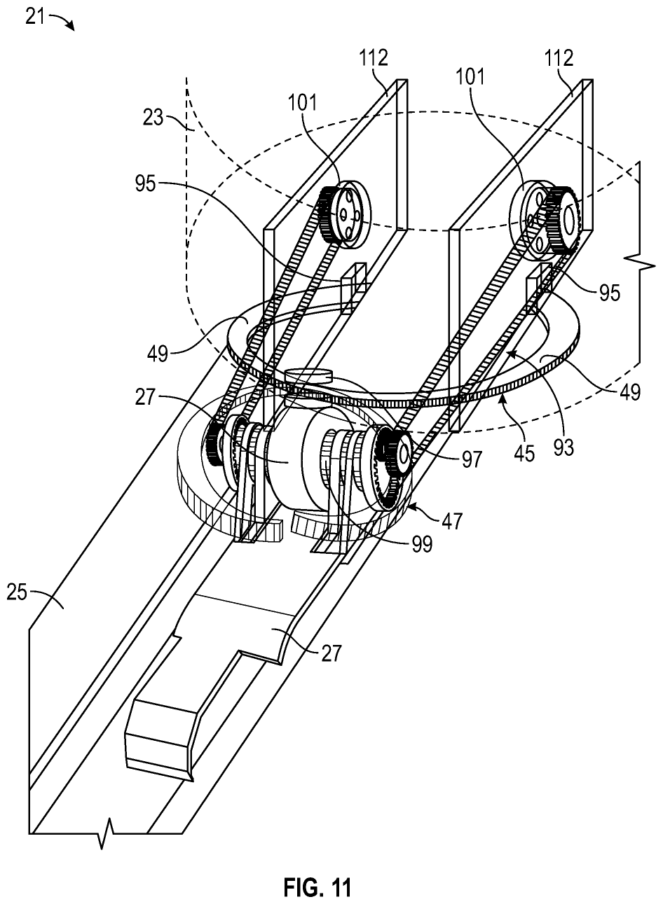

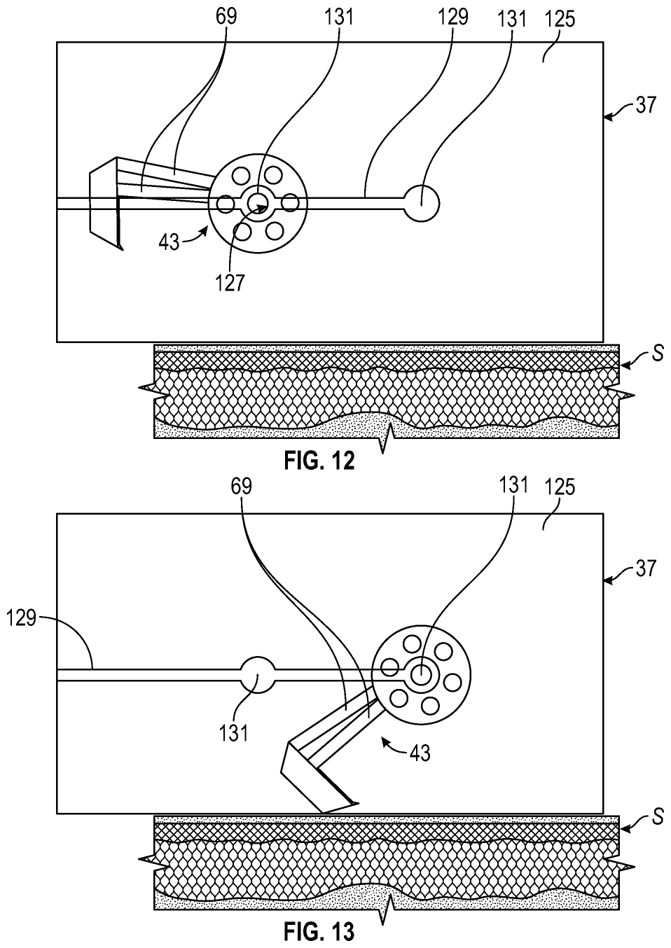

Dermatoscope 21 includes a crimping assembly 45 (FIG. 11) located relative to head 23 and handle 25 so that suitable actuation of manually actuatable controls 27 causes crimping assembly 45 to operatively connect to adhesive bandage-clip assembly 39 while disposed within cartridge 37. In one possible implementation, crimping assembly 45 has a bandage closure mechanism 47 connected to a pair of arms 49. Suitable actuation of bandage closure mechanism by manual controls will selectively move arms 49 inwardly in relation to the selected skin area and toward and adjacent to the biopsy incision previously performed by blade assembly 43, such inward movement crimping adhesive bandage-clip assembly 39 over the biopsy incision while head 23 is in the desired, operative position. Although any number of manually actuatable connections and controls may be associated with inward and outward movement of arms 49, one suitable implementation shown in FIG. 11 makes use of a rotatable thumb-wheel-style control linked to sides of cartridge 37.

Once arms 49 of crimping assembly 45 have been suitably advanced to crimp adhesive bandage-clip assembly 39 as desired by the practitioner, arms 49 of crimping assembly 45 may be selectively moveable outwardly, in which case arms 49 exit from operative interactions with cartridge 37 and thereby permit removal of cartridge 37 from head 23.

In view of the foregoing, dermatoscope 21 is a portable, self-contained apparatus, and is able to more efficiently perform a variety of dermatological procedures, such as first visualizing a selected skin area of interest, applying a suitable adhesive bandage to such skin area in anticipation of a further procedure, performing an incision in the selected skin area for purposes of a biopsy, closing the incision site by suitable operations by crimping the adhesive bandage-clip assembly overlying the selected skin area, collecting the biopsied skin area within cartridge 37, and selectively removing the biopsied material for analysis and the cartridge for subsequent disposal, such as in the sharps bin.

Adhesive bandage 41 has upper and lower surfaces defining a bandage area contained within and extending proximate to a distal side of cartridge 37. Lower surface of adhesive bandage 41 includes an adhesive contact surface which has a corresponding adhesion value. The adhesive contact surface opposes the selected skin area when the head is brought into the desired operative position.

Adhesive bandage 41 includes an aperture 89 formed therein, and upper surface of adhesive bandage 41 has a pair of flanges 91 disposed thereon and extending adjacent aperture 89. Edge portions of adhesive bandage-clip assembly 39 are received in engagement slots 95 formed on opposite sides or walls of cartridge 37. As such, engagement slots 95 retain adhesive contact surface 83 of adhesive bandage 41 at a predetermined, operative distance from the selected skin area of the patient. Adhesive bandage 41 has a predetermined flexibility in the direction orthogonal to its planar surfaces. The flexibility of adhesive bandage 41, along with the adhesion value of adhesive contact surface 83, and the operative distance at which adhesive bandage clip assembly 39 is held relative to the skin area, are all selected or determined so that when head 23 is either manually compressed toward the skin area or orthogonal forces otherwise applied toward the skin area onto adhesive bandage-clip assembly 39, such actions causing release of the adhesive bandage-clip assembly from cartridge 37 and adhere adhesive bandage 41 to the selected skin area thereby.

With regard to crimping assembly 45, in the illustrated implementations of FIGS. 1-11, the pair of arms 49 are rotatably mounted at least one pivot point 97 and extend distally from pivot point 97 to terminate at a distal end of dermatoscope 21 adjacent to respective ones of flanges 91 of adhesive bandage-clip assembly 39. Closure mechanism 47 of crimping assembly 45 makes use of at least one manually actuatable gear 99 connected to the pair of arms. Gear 99 is mounted relative to bandage closure mechanism 47 so actuation of gear 99 in a first direction corresponds to selectively moving arms 49 of crimping assembly 45 inwardly to push on opposing portions of flanges 91 of adhesive bandage-clip assembly 39. Conversely, actuation of gear 99 in a second direction moves arms 49 outwardly, releasing inward force from flanges 91.

The mounting subassembly 53 of blade assembly 43, in certain implementations, may comprise a pair of the mounting struts 69, each of the mounting struts 69 extending outwardly from respective convex sides 55 of blade 51 and transversely therefrom to terminate in connection ends 101. Connection ends 101, in turn, are rotatably connected at longitudinal sides 112 of cartridge 37. In turn, connection ends 101 of struts are operatively connected to a corresponding one of manually actuatable controls 27, so that blade 51 may be suitably moved or rotated relative to the skin area being visualized by head 23 to perform desired dermatological procedures, such as desired incisions for biopsy.

Blade 51 may assume a variety of characteristics as required for the biopsies to be performed therewith.

In the illustrated implementations, blade 51 has an open-boat shape characterized by a blade width, a blade length, and a blade depth. Suitable ranges of values for such blade width, blade length, and blade depth have been found to range as follows: width of 1-10 mm, length of 3-30 mm, and depth of 1-10 mm respectively. A greater range is possible as a function of the size of head 23. The depth of biopsy or excision which can be obtained with the boat shaped blade is determined by several factors, as set forth immediately below.

In implementations such as those illustrated in FIGS. 1-13, blade 51 may be considered to rotate about a mounting axis, as shown, which, in turn, is located at an operative distance from the skin area to be biopsied. Furthermore, head 23 contacts the skin at a corresponding contact distance, as shown. The operative distance, in this implementation, is greater than the contact distance, such that, when head 23 is appropriately positioned in operative contact with the skin, the difference between the operative distance through which the blade swings or rotates, and the contact distance at which head 23 is held forms a difference corresponding to the desired incision depth.

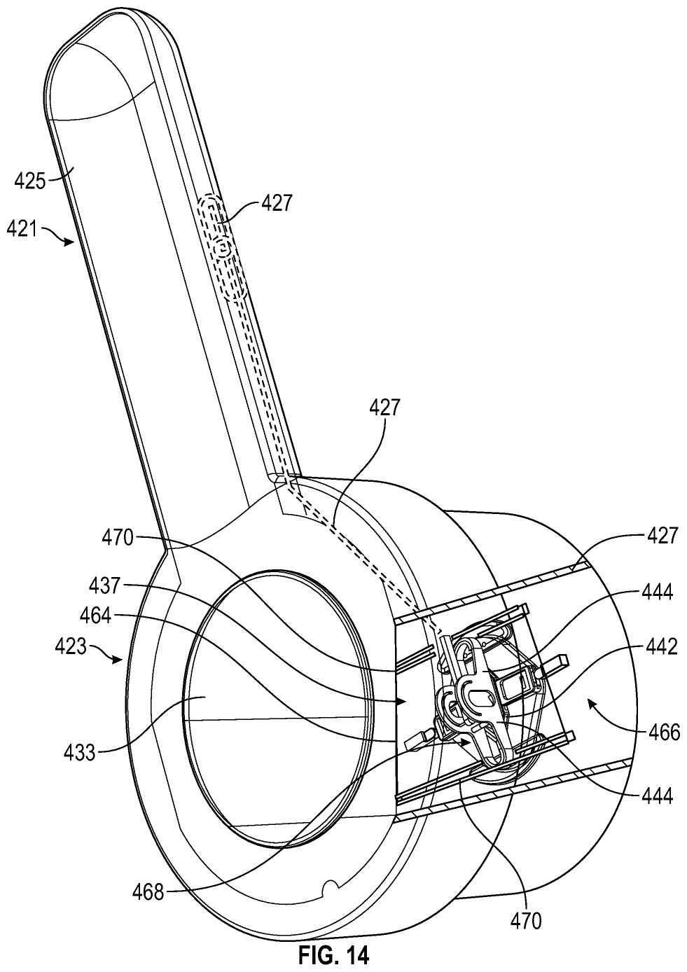

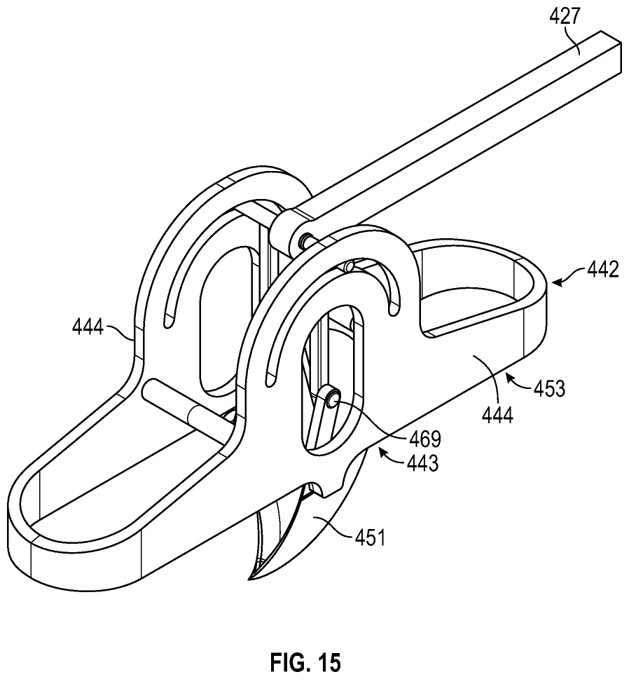

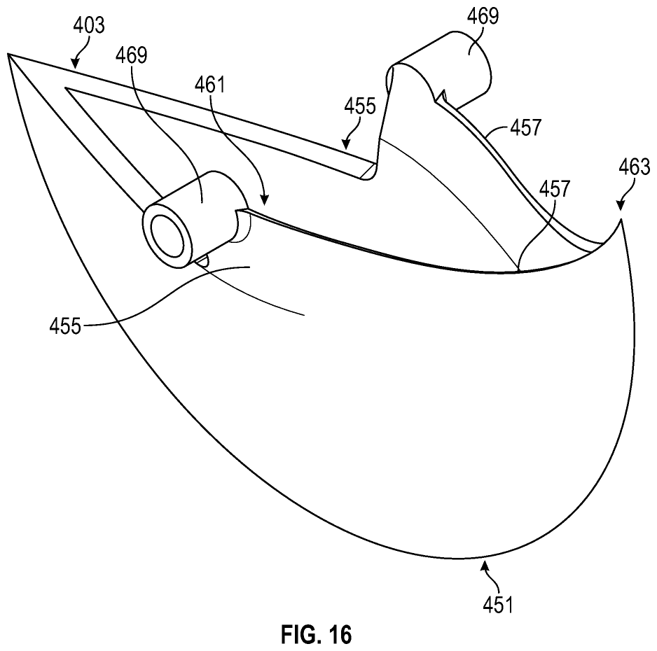

In another implementation of the device, as described and illustrated in FIGS. 14-20, the rotation of the boat shaped blade also occurs about a fixed point axis extending through struts 469. In such implementation, the length of the blade 491 and a standoff height of the rotational axis from the skin's surface (similar to the aforementioned difference between operative distance and contact distance) are often the main factors for determining the depth of cut in this method of blade actuation. In one suitable configuration, a blade roughly 9 mm long, 3 mm wide, and 5 mm deep mounted with a stand off height of 1.5 mm will excise a 3.3 mm deep tissue specimen. The desired standoff height may be a function of the thickness of the wound clip, so the depth of the specimen obtained may differ in certain applications.

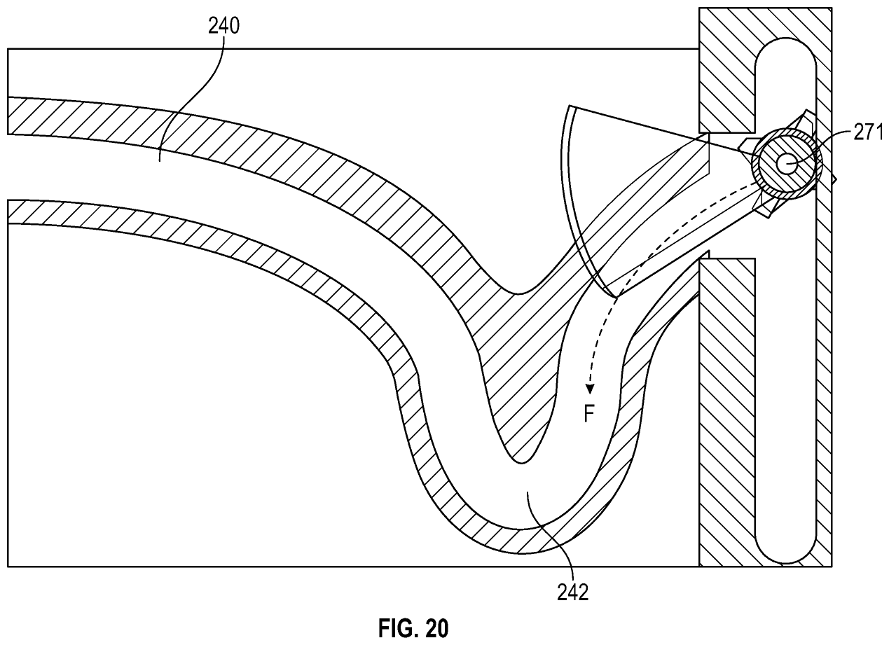

An alternative mechanism to the aforementioned blade rotation about a fixed pivot point is a moving pivot point at selected points or throughout the blade rotation, such moving pivot point induced by blade guides positioned at each lateral aspect of the blade track. A cross-sectional, side elevational view of one implementation of such a blade guide is seen in FIG. 20. In this embodiment, a slider shown at the right of the image has a blade assembly and associated pivot axis 271 therein, and is located adjacent to a track illustrated on the left to permit translation of the blade assembly. As the guide moves along the tract, it pushes the blade, forcing the blade through the track and altering or guiding the pivot point 271 as the blade rotates. This guide mechanism permits the device to utilize both the boat shaped blade as well as other geometries to obtain deeper tissue samples without necessitating a longer cut.

In addition, having convex edges 57 extend from the front and rear ends of the open-boat configuration to form a mathematical ellipse has been found suitable for certain applications.

Still further, a 1-to-3 ratio of blade width to blade length has been found suitable, with such values being selected from the group consisting of 1 mm×3 mm, 2 mm×6 mm, 3 mm×9 mm, 4 mm×12 mm, 5 mm×15 mm, 2.5 mm×7.5 mm, and 3.5 mm×10.5 mm to represent the predicted most commonly used sizes. Larger sizes can be produced as a function of the maximum dimensions of the device head. It is foreseen that multiple depths may be desirable for a given length to width ratio, and a suitable mechanism that modulates the stand off height of the fixed pivot point for the blade rotation may allow significant versatility in this fashion.

It is likewise within the scope of this disclosure to provide different blade geometries for blade 51 in different cartridges 37, thereby creating a set of disposable cartridges 37. The set of cartridges 37 may be appropriately labeled with blade dimensions having associated clinical characteristics to enable a practitioner to select a certain one of the set of cartridges to perform an associated biopsy excision with the corresponding clinical characteristics of such selected cartridge. The sample collected therefrom may be suitably removed from the cartridge and such cartridge again may thereafter be disposed of, as referenced previously.

It is likewise desirable, in certain implementations, for a manually actuatable control 27 to be interconnected so as to form a failsafe mechanism. For example, controls 27 may be configured so that one of the manually actuatable controls cannot be operated prior to a prerequisite actuation of another of the manually actuatable controls 27. In one suitable implementation, the failsafe mechanism inhibits rotation of blade assembly 43 prior to manual actuation of the control corresponding to application of the adhesive bandage-clip assembly. In this way, there is no premature actuation or rotation of blade 51 to either spoil the adhesive bandage-clip assembly 41 associated with a given cartridge 37, nor inadvertently enter the skin area of the patient prior to adhesion of bandage 41 to the desired skin area. Similarly, it would be preferable if the user were unable to initiate crimping of the bandage-clip prior to the blade removing the specimen. One manual actuation intended to remain independent from sequential events is the release of the bandage-clip assembly. The ability to eject the bandage-clip at any time enables the user to course correct in between any major step of the process to account for the inherent variabilities of clinical practice.

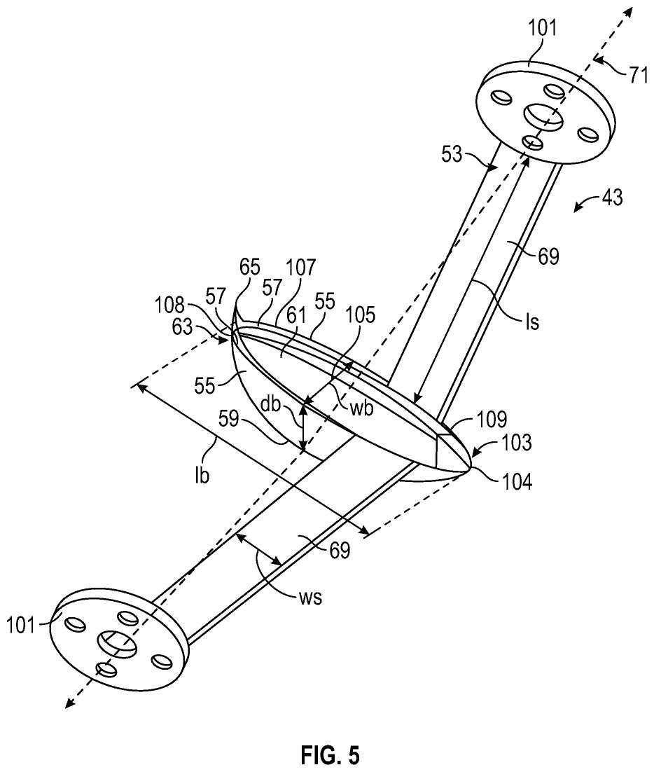

Blade assembly 43 may be associated with a maximum width wb (FIG. 5). Such maximum width wb may be located at a corresponding longitudinal location 105 between the forward and rear ends of blade 51. As such, convex edges 57 may have forward edge portions 107 extending between longitudinal location 105 at the maximum width wb toward forward tip 65. Furthermore, convex edges 57 may have rear edge portions 109 extending between longitudinal location 105 of maximum width wb and a rear tip 104.

The significance of such geometry is such that forward edge portions 107, in suitable implementations, are longer than rear edge portions 109 and thereby impart longitudinal tension on the selected skin area during rotation of blade assembly 43 during performance of biopsy incision.

Referring again to adhesive bandage-clip assembly 39, aperture 89 therein has a pair of spaced, opposing aperture edges 113 extending between upper and lower planar surface 42 of adhesive bandage 41. Aperture edges 113 extend arcuately relative to each other such that aperture 89 has an elongated form. The elongated form, in turn, is sized so that, after separation of the adhesive bandage onto the selected skin area, while head 23 is in the desired operative position, blade assembly 43 does not contact aperture edges 113 during rotation of blade assembly 43 between its stowed position and into its operative position during which biopsy is performed.

Adhesive bandage 41 may assume any number of forms and be formed of various suitable materials associated with the dermatological procedure to be performed. For example, certain portions of adhesive bandage 41 may comprise absorptive material, such as on lower planar surface 44 and sized and extended along aperture edges to optimally absorb excretions, blood, or other fluids associated with biopsy performance.

Adhesive bandage-clip assembly 39 includes a bandage clip 117 secured to upper planar surface 42 and having greater rigidity than the lower planar adhesive contact surface of bandage 41. Bandage clip 117 may extend orthogonally from upper planar surface 42 and proximate to aperture 89. Flanges 91 have flange portions oriented outwardly to selectively engage arms 49 of crimping assembly 45 when crimping assembly 45 is actuated by a corresponding one of manually actuatable controls 27. To that end, flanges 91 may be configured in a variety of ways, such as by including engagement portions 118 as illustrated, extending radially outwardly from aperture edges 113 or may comprise a pair of lips 119 extending proximate to aperture edges 113 and orthogonally from upper surface 42 of bandage 41. As illustrated, in certain implementations, flanges 91 may have both such structures to encourage movement of the underlying adhesive bandage 41 to cover the biopsy site thereby upon advancement of arms 49 of crimping assembly 45.

In still further implementations, the bandage-clip assembly 39 or bandage 41 may be equipped with opposing, engageable, interlocking portions 121 secured relative to each other so as to encourage wound closure, eversion, or other desirable closure attributes when elongated aperture 89 has been closed by crimping assembly 45.

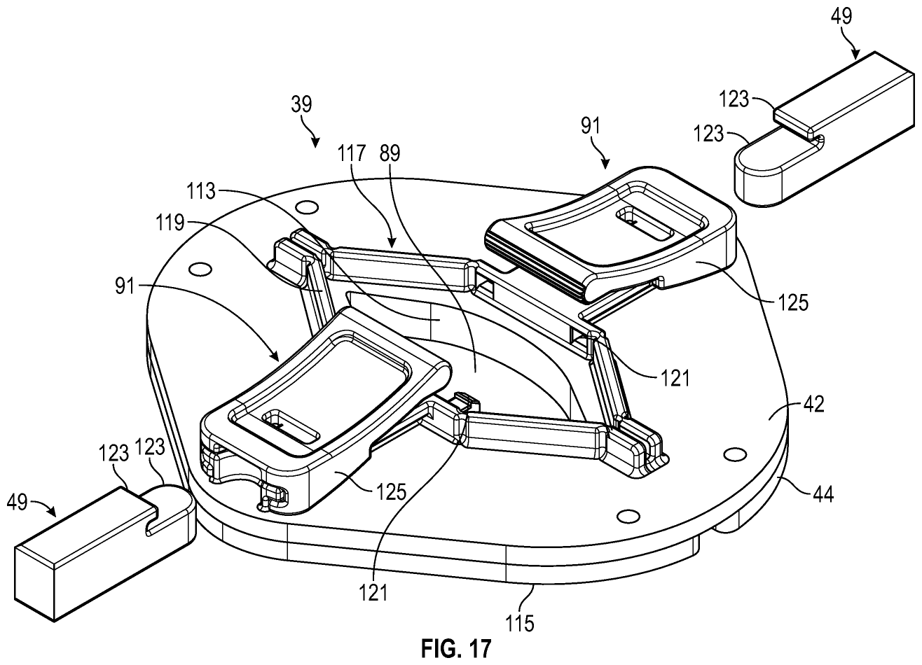

Still further implementations of adhesive bandage-clip assembly 39 are possible, such as the implementation shown in FIG. 17. In such implementation, arms 49 terminate in respective pairs of fingers 123. Flanges 91 include both the engagement portions extending radially outwardly from aperture edges as well as a pair of lips 119 extending proximate to such aperture edges. In addition, flanges 91 include sliders 125 independently slidable relative to lips 119. Configuration of fingers 123 in relation to the engagement portions of flanges 91 and lips 119 are such that one of the fingers 123 selectively engages the opposing, engagement portions of the flanges 91 and another of the fingers 123 thereafter selectively engages opposing portions of sliders 125. In this manner, lips 119 are advanced to pull the aperture 89 of bandage 41 by contact between the first of the fingers and opposing engagement portions of flanges 91 but thereafter sliders 125 advance by the other of the fingers 123 to impart additional closure force, such additional force including an upward component orthogonally away from the biopsy site and thereby promote eversion at the incision site. Inwardly oriented, opposing surfaces of sliders 125 may be equipped with interlocking features to further impart closing force to the adhesive bandage and promote eversion.

FIGS. 12 and 13 show partially schematic views of another suitable implementation of a dermatoscope cartridge according to the present disclosure. In particular, cartridge 37 includes respective longitudinal sides 125. Mounting subassembly 53 of blade assembly 43 is suitably connected not only to longitudinal sides as disclosed with reference to FIGS. 1-11, but at two longitudinal locations 131. Between the two respective longitudinal locations 131 are deployment channels 129 interconnecting such longitudinal locations. Mounting subassembly 53 is movable by being operatively connected to a suitable one of the manually actuatable controls and can thus be translated between the two different longitudinal locations 131. Such configuration allows mounting subassembly 53 to remain out of view when at one of the locations 131 and then moved into operative position by advancing to the other one of longitudinal locations 131, as shown in FIG. 13.

Referring now to FIGS. 14-16, still another suitable implementation of a dermatoscope is shown, with corresponding 400-series, reference numbers similar to structures shown in the embodiment of FIGS. 1-11. Accordingly, a dermatoscope 421 includes a head 423 at an end of an elongated handle 425, with the various features of dermatoscope 421 operable by manually actuatable controls 427. The subassembly 453 shown in FIGS. 14-16 includes a mounting frame 442 and a pair of mounting struts 469. Mounting frame 442 has longitudinal sides 444, and mounting struts 469 are located at respective convex sides 455 of blade 451.

The overall open-boat shaped structure of blade 451 is similar to that previously described. Mounting struts 469 are mounted to respective longitudinal sides of mounting frame 442 and rotatable relative thereto to the operative position in response to actuation of a corresponding one of manually actuatable controls 427. Cartridge 437 has an outer wall receivable within head 423 of dermatoscope 421 and an inner wall defining central and edge zones 468, 466, respectively therein. Central zone 468 is visible through viewing opening through lens 433 whereas edge zone 466 is not visible through the viewing opening and lens 433.

Mounting subassembly 453 further includes a rack 464 with a pair of rails 470 extending between edge zone 466 and central zone 468. Mounting frame 442 is slidably mounted to rack 464. In this way, mounting frame 442 is positionable by sliding, in response to a first actuation of manually actuatable controls 427, to position mounting frame 442 and blade 451 thereof in edge zone 466. Edge zone 466 corresponds to the stowed position of blade assembly 43 with reference to the embodiments shown in FIGS. 1-11.

Mounting frame 442 is likewise slidable, in response to a second actuation of suitable manually actuatable controls 427, to thereby position mounting frame 442 and blade 451 associated therewith in central zone 468, which central zone corresponds to the second operative position as discussed previously. In such operative position, blade 451 is thereafter rotatable to contact the desired skin area to perform the biopsy incision thereof.

Still further implementations of the dermatoscope of the present disclosure are shown with reference to FIGS. 18 and 19. In such implementations, adhesive bandage-clip assembly 339 comprises a pair of interlockable staples 346. Crimping assembly 345 comprises a rotatable crimping ring transversely mounted on head 323. Arms 349 of crimping assembly 345 in the illustrated implementation of FIGS. 18 and 19 comprise cam portions 350 extending radially inwardly from rotatable crimping ring 352. Crimping ring 352, in turn, is rotatable between a first position shown in FIG. 18 in which arms 349 are disengaged from staples 346 and a second position shown in FIG. 19 in which arms are engaged with staples 346 to crimp the adhesive bandage-clip assembly 339 over the biopsy incision site.

Referring to still further possible implementations, FIG. 20 shows another suitable configuration of the blade assembly 243 relative to cartridge 37. In this case, a pair of guide rails 240 are defined on two sides, respectively, of the corresponding head 23 to ensure parallel support. Guide rails 240 have lower portions 242 which corresponds to the operative portion of the blade's actuation. As such, mounting subassembly of the blade assembly 243 is movable to position blade mounting axis 271 in the lower portions 242 of guide rails 240. In such position, the mounting axis 271 is spaced from contact with the desired skin area by the predetermined operative distance through which blade 251 rotates upon suitable actuation by manual controls 27, as explained previously. The significant utility of this blade guide mechanism is the creation of a moving pivot throughout the cut, which allows the blade to obtain deeper specimens without significantly increasing the length of the cut.

Another suitable implementation of the device, contains all of these described mechanics for cutaneous biopsy or excision, utilizing the same cartridge, blade, and bandage-clip assemblies described in this section, but has a simpler array of optical features appropriate for a clinician who is not trained in dermoscopy. Current implementations of cross-polarized dermoscopy require a polarized light source, and a polarized analyzer oriented at 90 degrees between the skin and viewing lenses. Many also utilize higher quality lenses, in groups, to permit focusing capabilities and enhance optical characteristics. These models without cross polarized visualization capabilities may more accurately be described as surface microscopes and have the benefits of being lighter, less expensive, and make the device's capabilities more accessible to a broader spectrum of clinicians. The device characteristics described herein should be interpreted as applicable even if the optical configuration incorporated in that design is more accurately described as an illuminated magnifier, or microscope, rather than that of a cross polarized dermatoscope. Versions of the described device with full high quality cross polarized optical capabilities, versions with minimal optical features and lighting options, as well as versions in-between, can all be made to offer the same mechanistic biopsy or excisional capabilities.

The dermatoscope of this disclosure may also be used to perform cutaneous excisions, wherein the elliptical biopsy captured by the boat shaped blade fully removes the lesion of interest with sufficient deep and lateral margins that histopathological clearance might reasonably be obtained. Cutaneous lesions requiring complete removal can range from less than 1 mm in size, to upper ranges that would vastly exceed the size of the device. So long as the blade sizes are capable of removing the entire lesion of interest with an appropriate surgical margin, the device could foreseeably perform excisional biopsies and cutaneous excision as well.

The term “dermatoscope,” as used herein, includes devices regardless of the types of lenses, optics, and light sources associated therewith. The term dermatoscope thus includes but is not limited to a cross-polarized dermatoscope. Furthermore, when referring to the various procedures as biopsy, incision, removal, or excision, it is understood that the disclosed and claimed dermatoscopes are capable of performing any of the foregoing and still other procedures involving contact between the blade and the skin or tissue of a patient. Accordingly, reference to a biopsy or to an incision in terms of what the disclosed dermatoscope is acting upon does not exclude use for excision and removal, and conversely.

It will be appreciated that still further implementations, variations, and modifications to the foregoing remain within the spirit and scope of the present invention and are likewise to be considered part of this disclosure, the scope of which is only as set out by the claims appended hereto and equivalents thereof.

What is claimed is:

1. A biopsy-capable dermatoscope for a practitioner to use on a patient, the dermatoscope comprising:
a head configured to be placed in operative contact with the patient's skin for skin visualization and biopsy procedures;
a handle extending from the head for grasping by the practitioner, the handle having manually actuatable controls for the visualization and the biopsy procedures;
a viewing opening disposed in the head and defining a viewing area, the viewing opening having at least one lens disposed across the viewing opening and a light source located relative to the viewing opening to illuminate a selected skin area of the patient when the head is brought into a desired, operative position on the patient;
a disposable cartridge removably inserted into the head;
an adhesive bandage-clip assembly disposed within the cartridge and positioned in operative proximity to the selected skin area when the head is brought into the desired operative position on the patient, the adhesive bandage-clip assembly having an adhesive bandage releasably secured relative to the cartridge to selectively separate the adhesive bandage from the head and onto the selected skin area when the head is in the desired operative position on the patient;
a blade assembly configured to perform a biopsy excision in response to manual actuation of one of the controls, the blade assembly mounted within the cartridge and rotatable, in response to manual actuation of one of the controls, when the head is in the desired operative position, the blade assembly being movable from a first, stowed position outside a majority of the viewing area defined by the viewing opening, and a second, operative position in contact with the skin area to perform a biopsy excision thereof;
a crimping assembly disposed in operative proximity to the adhesive bandage-clip assembly, the crimping assembly having a bandage closure mechanism and a pair of arms, one of the manually actuatable controls connected to the closure mechanism to selectively move the arms inwardly in relation to the selected skin area, and toward and adjacent to the biopsy incision to crimp the adhesive bandage-clip assembly over the biopsy incision while the head is in the desired, operative position;
wherein the arms of the crimping assembly are selectively movable outwardly after the crimping of the adhesive bandage-clip assembly and do not impinge upon removal of the cartridge from the head; and
wherein the head, the handle, and the removably inserted cartridge comprise a portable, self-contained apparatus.

2. The biopsy-capable dermatoscope of claim 1,
wherein the blade assembly comprises a blade and a mounting subassembly for the blade;
wherein the blade is configured into an open-boat shape, the open-boat shape having a pair of convex sides extending upwardly from a bottom portion to define a corresponding pair of convex, upper edges, the bottom, sides, and edges defining an open volume to collect a sample after completing the biopsy incision;
wherein the upper edges are sufficiently sharp to comprise respective, cutting edges, the upper edges meeting at a forward end, the forward end including a forward portion configured to be sufficiently sharp to form a cutting tip;
wherein the mounting subassembly comprises at least one mounting strut rotatable about an associated transverse, blade mounting axis, the mounting strut connected to the blade to position the forward end of the blade at a predetermined operative distance from the mounting axis, whereby the blade rotates relative to the blade mounting axis at the predetermined operative distance;
wherein the head includes a longitudinal axis and has longitudinally spaced ends comprising a viewing end and an operative end opposite the viewing end, the operative end terminating in inner and outer concentric edges to form a contact ring having an associated contact surface to make contact with the selected skin area when the head is brought into the desired, operative position on the patient;
wherein the contact surface of the contact ring is spaced from the blade mounting axis by a contact distance;
wherein the predetermined operative distance through which the blade rotates is greater than the contact distance between the blade mounting axis and the contact surface of the head to define a corresponding difference; and
wherein the difference between the predetermined operative distance and the contact distance corresponds to an incision depth;
whereby, in response to actuation of a corresponding one of the controls to move the blade from the stowed position to the operative position, the blade is rotatable to perform the biopsy incision at the incision depth.

3. The biopsy-capable dermatoscope of claim 2,
wherein the cartridge has a longitudinal axis defined therein and includes respective, opposite, longitudinal sides;
wherein the mounting subassembly and the cartridge are connected to each other at the longitudinal sides at a first, longitudinal location along the longitudinal axis of the cartridge.

4. The biopsy-capable dermatoscope of claim 3,
wherein the longitudinal sides of the cartridge have respective, longitudinally extending deployment channels formed therein, the deployment channels having two receiving locations, the first receiving location defined at the first longitudinal location, and the second receiving location defined at a second longitudinal location;
wherein the mounting subassembly is movably mounted with respect to the channels and operatively connected to one of the manually actuatable controls to be translatable between the two different longitudinal locations;
wherein the longitudinal location closer to the viewing end corresponds to the stowed position of the blade assembly and the longitudinal location closer to the operative end corresponds to the operative position of the blade assembly.

5. The biopsy-capable dermatoscope of claim 2,
wherein the mounting subassembly further comprises a mounting frame and a pair of the mounting struts, the mounting frame having longitudinal sides, the pair of mounting struts located at respective convex sides of the blade; and
wherein the mounting struts are mounted to respective longitudinal sides of the mounting frame and rotatable relative to the mounting frame to the operative position in response to actuation of a corresponding one of the manually actuatable controls.

6. The biopsy-capable dermatoscope of claim 5,
wherein the cartridge has an outer wall receivable within the head of the dermatoscope and an inner wall defining central and edge zones within the inner wall, the central zone being visible through the viewing opening and the edge zone being not visible through the viewing opening;
wherein the mounting subassembly further comprises a rack with a pair of rails extending from the edge zone of the cartridge to the central zone of the cartridge;
wherein the mounting frame is slidably mounted to the rack;
wherein the mounting frame is slidable, in response to a first actuation of a suitable one of the manually actuatable controls, to position the mounting frame and the blade mounted thereto in the edge zone, which position corresponds to the stowed position of the blade assembly; and
wherein the mounting frame is slidable, in response to a second actuation of a suitable one of the manually actuatable controls, to position the mounting frame and the blade mounted thereto in the central zone, which position in the central zone corresponds to the second operative position in which the blade is rotatable to contact the skin area to perform the biopsy incision thereof.

7. The biopsy-capable dermatoscope of claim 1,
wherein the cartridge has a distal side opposing the selected skin area of the patient when the head is brought into the desired operative position on the patient;
wherein the adhesive bandage-clip assembly comprises planar upper and lower surfaces having edge portions defining a bandage area contained within and extending across the distal side of the cartridge, the lower surface including an adhesive contact surface having a corresponding adhesion value, the adhesive contact surface opposing the selected skin area when the head is brought into the desired operative position;
wherein the adhesive bandage has an aperture formed therein and a pair of flanges extending adjacent the aperture on the upper surface of the adhesive bandage;
wherein the cartridge includes opposite walls having engagement slots defined therein and located proximate to the distal side of the cartridge, engagement slots sized and located to releasably receive the edge portions of the bandage-clip assembly therein to retain the adhesive contact surface at a predetermined, operative distance from the selected skin area of the patient;
wherein the adhesive bandage-clip assembly has a predetermined flexibility orthogonal to the planar surfaces thereof;
wherein the flexibility, the adhesion value, and the operative distance are selected to cause release of the adhesive bandage-clip assembly from the cartridge in response to selective application of orthogonal force toward the selected skin area.

8. The biopsy-capable dermatoscope of claim 7,
wherein the dermatoscope has opposite proximal and distal ends, the handle extending from the proximal end toward the distal end, the head located at the distal end;
wherein the pair of arms of the crimping assembly is rotatably mounted at least one pivot point and extends distally from the pivot point to terminate at distal ends adjacent to respective ones of the pair of flanges of the adhesive bandage-clip assembly;
wherein the bandage closure mechanism of the crimping assembly comprises at least one manually actuatable gear connected to the pair of arms; and
wherein the gear is mounted relative to the bandage closure mechanism so that actuation of the gear in a first direction corresponds to selectively moving the arms inwardly to crimp the adhesive bandage-clip assembly and actuation of the gear in a second direction corresponds to selectively moving the arms outwardly after the crimping.

9. The biopsy-capable dermatoscope of claim 2,
wherein the mounting subassembly of the blade assembly comprises a pair of the mounting struts, each of the mounting struts extending outwardly from respective sides of the blade and transversely therefrom to terminate in connection ends at respective longitudinal sides of the cartridge;
wherein the connection ends of the struts are operatively connected to a corresponding one of the manually actuatable controls.

10. The biopsy-capable dermatoscope of claim 2, wherein the open-boat shape of the blade comprises a blade with a blade width of 3 mm, a blade length of 9 mm, and a blade depth of 5 mm.

11. The biopsy-capable dermatoscope of claim 2,
wherein the blade has a rear end opposite the forward end, the forward and rear ends terminating in corresponding forward and rear tips; and
wherein the convex edges extend between the rear and forward tips and define a mathematical ellipse having the blade length equal to three times the blade width.

12. The biopsy-capable dermatoscope of claim 10,
wherein the blade width has a maximum width at a corresponding, longitudinal location between the forward and rear ends of the blade;
wherein the convex edges have forward edge portions extending between the longitudinal location of the maximum width and the forward tip, the convex edges having rear edge portions extending between the longitudinal location of the maximum width and the rear tip; and
wherein the forward edge portions of the convex edges are longer than the rear edge portions of the convex edges to impart longitudinal tension on the selected skin area during rotation of the blade assembly during the performing of the biopsy incision.

13. The biopsy-capable dermatoscope of claim 7,
wherein the aperture is defined by a pair of spaced, opposing aperture edges extending between the upper and lower planar surfaces of the adhesive bandage, the aperture edges extending arcuately relative to each other to define an elongated opening therebetween;
wherein the elongated opening is sized so that, after the separation of the adhesive bandage onto the selected skin area, while the head is in the desired, operative position, the blade assembly does not contact the aperture edges during rotation of the blade assembly between the stowed position and the operative position;
wherein the bandage comprises absorptive material on the lower surface extending along the aperture edges;
wherein the adhesive bandage-clip assembly further includes a bandage-clip secured to the upper surface of the adhesive bandage, the bandage-clip having greater rigidity than the adhesive contact surface, the bandage-clip located and extending proximate to the elongated opening;
wherein the flanges include at least one structure selected from the group consisting of engagement portions extending radially outwardly from the aperture edges and a pair of lips extending proximate to the aperture edges and orthogonally from the upper surface of the bandage;
wherein the flanges have flange portions oriented outwardly to selectively engage the arms of the crimping assembly when the crimping assembly is actuated by a corresponding one of the manually actuatable controls;
wherein the flange portions are located relative to the aperture edges to move the corresponding edges of the aperture toward each other to close the elongated opening of the bandage upon a sufficient amount of advancement of the arms of the crimping assembly, and whereby the biopsy site is covered by the adhesive bandage.

14. The biopsy-capable dermatoscope of claim 13, wherein the bandage has edges with opposing, engageable, interlocking portions secured relative to each other when the elongated opening has been closed.

15. The biopsy-capable dermatoscope of claim 13,
wherein the flanges comprise the engagement portions extending radially outwardly from the aperture edges and the pair of lips extending proximate to the aperture edges;
wherein the flanges further comprise sliders independently slidable relative to the lips between a first, disengaged slider position and a second engaged slider position radially inwardly from the disengaged slider position;
wherein the sliders have radially outwardly facing slider positions; and
wherein the arms terminate in respective pairs of fingers, one of the fingers oriented and configured to selectively engage the engagement portions of the flanges and another of the fingers oriented and configured to selectively engage the outwardly facing portions of the sliders.

16. The biopsy-capable dermatoscope of claim 15, wherein, in response to advancing the arms by actuation of corresponding controls, the lips are advanced to reduce the size of the elongated opening of the bandage by contact between the first finger and the opposing engagement portions of the flanges and, thereafter, the sliders are advanced to impart force upon the bandage upwardly relative to the planar surfaces of the bandage to promote eversion of the incision site.

17. The biopsy-capable dermatoscope of claim 1, wherein the manually actuatable controls are interconnected to form at least one fail-safe mechanism inhibiting the actuation of at least one of the manually actuatable controls prior to a prerequisite actuation of another of the manually actuatable controls.

18. The biopsy-capable dermatoscope of claim 17, wherein the fail-safe mechanism is configured to inhibit rotation of the blade assembly prior to manual actuation of the control corresponding to the release of the adhesive bandage-clip assembly.

19. The biopsy-capable dermatoscope of claim 1,
wherein the disposable cartridge comprises one of a set of disposable, removably insertable cartridges, each of the cartridges containing therein a corresponding blade assembly having a set of blade dimensions with associated clinical characteristics;
wherein the set of blade dimensions of at least a first one of the set of cartridges differs from the set of blade dimensions of at least a second one of the set of cartridges, the first and second cartridges having respective indicia associated therewith to identify the associated, different blade dimensions; and
whereby the disposable cartridge removably inserted into the head is selectable from the set of cartridges, including the cartridges having the indicated set of blade dimensions to perform the biopsy incision associated with the corresponding clinical characteristics.

20. The biopsy-capable dermatoscope of claim 19, wherein the set of cartridges may comprise cartridges containing blade assemblies having blade-width-to-blade-length values selected from the group consisting of 1 mm×3 mm, 2 mm×6 mm, 3 mm×9 mm, 4 mm×12 mm, 5 mm×15 mm, 2.5 mm×2.75 mm, and 3.5 mm×10 mm.

21. The biopsy-capable dermatoscope of claim 4, wherein the head comprises guide rails on two sides thereof;
wherein the guide rail has a lower portion; and
wherein the mounting subassembly of the blade assembly is movable to position the blade mounting axis in the lower portion of the guide rail to define the predetermined operative distance through which the blade rotates.

22. The biopsy-capable dermatoscope of claim 1,
wherein the adhesive bandage-clip assembly comprises a pair of interlockable staples;
wherein the crimping assembly comprises a rotatable crimping ring transversely mounted on the head;
wherein the arms of the crimping assembly comprise cam portions extending radially inwardly from the rotatable crimping ring; and
wherein the crimping ring is rotatable between a first position in which the arms are disengaged from the staples and a second position engaged with the staples to crimp the adhesive bandage-clip assembly.

23. A method of performing a biopsy on skin of a patient, the method comprising the steps of:
bringing a head of a dermatoscope into an operative position relative to a skin area of interest;
while maintaining the head in the same operative position as the previous step, visualizing the skin area through an opening within the head of the dermatoscope;
while maintaining the head in the same operative position as used for the previous step of visualizing the skin area, applying a suitable adhesive bandage from a stored position within the head to the desired skin area in anticipation of one or more dermatological procedures thereafter;
while maintaining the head in the same operative location as the foregoing steps, actuating a blade housed within the head to perform a biopsy incision in the selected skin area;
while maintaining the head in the same operative position as the foregoing steps, collecting the biopsied skin sample in the cartridge after performance of the biopsy; and
after performance of the biopsy, closing an aperture in the adhesive bandage over the incision.

24. The method of claim 23, further comprising the step of inserting a disposable cartridge into the head of the dermatoscope, the disposable cartridge having the adhesive bandage and the blade assembly for performing the biopsy procedure disposed in the cartridge; and
after performance of the biopsy procedure, removing the disposable cartridge from the head of the dermatoscope.

25. The method of claim 24, further comprising the steps of
selecting one disposable cartridge having a blade assembly therein with associated clinical characteristics from an available set of disposable cartridges having blade assemblies of different, respective associated clinical characteristics; and
performing the biopsy with the blade assembly of the selected cartridge to achieve the associated clinical characteristics.
